(19) United States
(12) Patent Application Publication
(10) Pub. No.: US 2017/0296097 A1
(43) Pub. Date: Oct. 19, 2017

(54) SYSTEMS AND METHODS FOR ESTIMATING VOLUME AND DENSITY

(71) Applicants: Jessica Li Walling, Atlanta, GA (US); Robert Richard Walling, III, Atlanta, GA (US)

(72) Inventors: Jessica Li Walling, Atlanta, GA (US); Robert Richard Walling, III, Atlanta, GA (US)

(21) Appl. No.: 15/477,262

(22) Filed: Apr. 3, 2017

Related U.S. Application Data
(63) Continuation of application No. 62/323,726, filed on Apr. 17, 2016.

(51) Int. Cl. A61B 5/107; A61B 5/00; A41C 3/04

(52) U.S. Cl. CPC A61B 5/1073; A61B 5/7278; A61B 5/6804; A61B 5/4866; A41C 3/04; A61B 5/4519; A61B 5/4288; A61B 5/0002; A61B 5/486

(57) ABSTRACT

Methods, systems, and computer readable medium are disclosed herein comprising detecting a first tension; detecting a second tension; determining a difference between the first tension and the second tension; and estimating a volume change based at least in part on the determined difference.

This application claims the priority benefit under 35 U.S.C. §119(e) from Provisional Application No. 62/323,726 filed Apr. 17, 2016, which is incorporated herein by reference in its entirety.

### SUMMARY

It is to be understood that both the following general description and the following detailed description are exemplary and explanatory only and are not restrictive, as claimed. Methods, systems, and computer readable medium are disclosed herein comprising detecting a first tension; detecting a second tension; determining a difference between the first tension and the second tension; and estimating a volume change based at least in part on the determined difference.

Methods, systems, and computer readable medium are disclosed herein comprising detecting a first tension; detecting a second tension; determining a difference between the first tension and the second tension; and estimating a density change based at least in part on the determined difference.

Methods, systems, and computer readable medium are disclosed herein comprising receiving a first tension; receiving a second tension; determining a difference between the first tension and the second tension; and estimating a volume change based at least in part on the determined difference.

Methods, systems, and computer readable medium are disclosed herein comprising receiving a first tension; receiving a second tension; determining a difference between the first tension and the second tension; and estimating a density change based at least in part on the determined difference.

Methods, systems, and computer readable medium are disclosed herein comprising detecting a first tension; transmitting the first tension to a user device; detecting a second tension; and transmitting the second tension to the user device, wherein the user device determines a difference between the first tension and the second tension, and wherein the user device estimates a volume change based at least in part on the determined difference.

Methods, systems, and computer readable medium are disclosed herein comprising detecting a first tension; transmitting the first tension to a user device; detecting a second tension; and transmitting the second tension to the user device, wherein the user device determines a difference between the first tension and the second tension, and wherein the user device estimates a density change based at least in part on the determined difference.

Methods, systems, and computer readable medium are disclosed herein comprising detecting a first tension; detecting a second tension; and determining completion of a repetition based at least in part on the detected tensions.

Methods, systems, and computer readable medium are disclosed herein comprising receiving a first tension; receiving a second tension; and determining completion of a repetition based at least in part on the detected tensions.

Methods, systems, and computer readable medium are disclosed herein comprising detecting a first tension; transmitting the first tension to a user device; detecting a second tension; and transmitting the second tension to the user device, wherein the user device determines completion of a repetition based at least in part on the detected tensions.

The above described and other features are exemplified by the following figures and detailed description.

### BRIEF DESCRIPTION OF THE DRAWINGS

FIG. 1 is an exemplary system.

FIG. 2 is an exemplary system.

FIG. 3 is an exemplary user interface.

FIG. 4 is an exemplary system.

FIG. 5 is an exemplary system.

FIG. 6 is an exemplary user interface.

FIG. 7 is a flow chart of an exemplary method.

FIG. 8 is a flow chart of an exemplary method.

FIG. 9 is a flow chart of an exemplary method.

FIG. 10 is a flow chart of an exemplary method.

FIG. 11 is a flow chart of an exemplary method.

FIG. 12 is a flow chart of an exemplary method.

FIG. 13 is a flow chart of an exemplary method.

FIG. 14 is a flow chart of an exemplary method.

FIG. 15 is a flow chart of an exemplary method.

### DETAILED DESCRIPTION

Reference throughout this specification to “exemplary embodiment” or “one embodiment” or “an embodiment” means that a particular feature, structure, or characteristic described in connection with the embodiment is included in at least one embodiment of the present invention. Thus, appearances of the phrases “in an exemplary embodiment” or “in one embodiment” or “in an embodiment” in various places throughout this specification are not necessarily all referring to the same embodiment.

Furthermore, the described features, structures, or characteristics may be combined in any suitable manner in one or more embodiments. In the following description, numerous specific details are provided, such as examples of user interfaces, screenshots, flow diagrams, etc., to provide a thorough understanding of embodiments of the invention. One skilled in the relevant art will recognize, however, that the invention can be practiced without one or more of the specific details, or with other methods, components, materials, etc. In other instances, well-known structures, materials, or operations are not shown or described in detail to avoid obscuring aspects of the invention.

“Or,” as used herein, except where noted otherwise, is inclusive, rather than exclusive. In other words, “or” is used to describe a list of alternative things in which one may choose one option or any combination of alternative options. For example, “A or B” means “A or B or both” and “A, B, or C” means “A, B, or C, in any combination.” If “or” is used to indicate an exclusive choice of alternatives or if there is any limitation on combinations of alternatives, the list of alternatives specifically indicates that choices are exclusive or that certain combinations are not included. For example, “A or B, but not both” is used to indicate use of an exclusive “or” condition. Similarly, “A, B, or C, but no combinations” and “A, B, or C, but not the combination of A, B, and C” are examples where certain combination of alternatives are not included in the choices associated with the list.

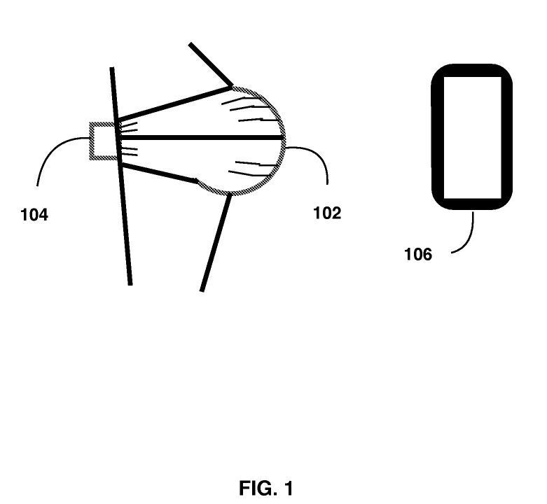

Referring now to FIG. 1, an exemplary system is illustrated. In an aspect, the system can comprise one or more articles of clothing (e.g., fabrics, bras, wraps, sleeves, supports, compression clothing, watches, shirts, pants, etc.) 102. In an aspect, the one or more articles of clothing 102 can comprise a computing device 104. In an aspect, the one or more articles of clothing 102 can comprise material. In an aspect, the computing device 104 can comprise one or more processors. In an aspect, the computing device 104 can detect tension related to the material of the one or more articles of clothing 102. In an aspect, the computing device 104 can be in communication with a sensor. In an aspect, the sensor can be used to detect tension. In an aspect, detecting tension can comprise detecting pressure with the computing device 104 and/or sensor. In an aspect, detecting tension can comprise detecting one or more circumferences with the computing device 104 and/or the sensor. In an aspect, the computing device 104 can comprise a transmitter. In an aspect, the transmitter can be a transceiver. In an aspect, the computing device 104 can be in communication with a user device 106. In an aspect, the computing device 104 can be in wireless communication with the user device 106. In an aspect, the wireless communication can be Wi-Fi, Bluetooth., etc. In an aspect, the computing device 104 can be in wired communication with the user device 106. In an aspect, the user device 106 can be a smart phone, tablet, laptop, desktop, wearable computing device, etc.

In an aspect, the computing device 104 can represent the detected tension using numbers, units, force, newtons, pounds-force, pounds, etc. In an aspect, the computing device 104 can transmit the detected tension to the user device 106. In an aspect, the computing device 104 can perform one or more evaluations (e.g., computations, etc.) using the detected tension and transmit the results of the one or more computations to the user device 106.

In an aspect, FIG. 1 can illustrate the one or more articles of clothing 102 detecting tension at a first state. As an example, the one or more articles of clothing 102 can be a bra 102. In the example, the computing device 104 can detect tension in the bra 102 when the wearer is in a pre-feeding state. In an aspect, the computing device 104 can transmit the detected tension to the user device 106, wherein the user device 106 can use the detected tension for one or more evaluations. In another aspect, the computing device 104 can perform one or more evaluations using the detect tension, wherein the computing device 104 can transmit the results of the one or more evaluations to the user device 106. In an aspect, the one or more evaluations can also use historical information. For example, historical information about the tension on the bra 102 can be used to determine that the current tension on the bra 102 indicates that a volume of milk in breasts of a user of the bra 102 is more, less, or substantially consistent with a typical and/or normal volume of milk. As an example, the current tension on the bra 102 can indicate a supply of milk lower or higher than is typical and/or normal. In response to the indication that the supply of milk is lower or higher than is typical and/or normal, the user device 106 can receive, create, and/or present a notification. In an aspect, the computing device 104 can create, transmit, and/or present the notification. The notification can be visual, audio, and/or tactile.

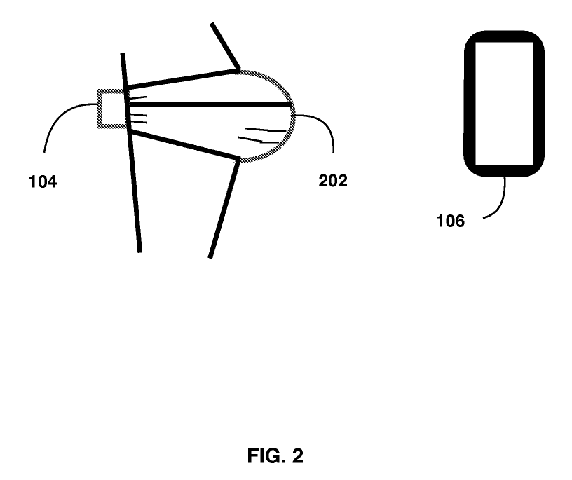

In an aspect, FIG. 2 can illustrate the one or more articles of clothing 202 detecting tension at a second state. As an example, the one or more articles of clothing 202 can be a bra 202. In the example, the computing device 104 can detect tension in the bra 202 when the wearer is in a post-feeding state. In an aspect, the computing device 104 can transmit the detected tension to the user device 106, wherein the user device 106 can use the detected tension for one or more evaluations. In another aspect, the computing device 104 can perform one or more evaluations using the detect tension, wherein the computing device 104 can transmit the results of the one or more evaluations to the user device 106. In an aspect, the one or more evaluations can also use historical information. For example, historical information about the tension on the bra 202 can be used to determine in the current tension on the bra 202 indicates that a volume of milk in breasts of a user of the bra 202 is more, less, or substantially consistent with a typical and/or normal volume of milk. As an example, the current tension on the bra 202 can indicate a supply of milk lower or higher than is typical and/or normal. In response to the indication that the supply of milk is lower or higher than is typical and/or normal, the user device 106 can receive, create, and/or present a notification. In an aspect, the computing device 104 can create, transmit, and/or present the notification. The notification can be visual, audio, and/or tactile. In an aspect, a user can track one or more diets and/or one or more activities. Diets can comprise food, drinks, nutritional supplements, medication, the like, and/or any combination of the foregoing. Activities can comprise workout routines, workout durations, aerobic activities, anaerobic activities, the like, and/or any combination of the foregoing. In an aspect, milk production based on volume change can be correlated to the one or more diets and/or the one or more activities for an individual user and/or for an aggregation of multiple users. A user can indicate a target milk production and/or a target change in milk production. A diet recommendation and/or an activity recommendation can be made based on the target milk production and/or the target change in milk production.

In an aspect, the detected tension at the first state can be compared with the detected tension at the second state. In an aspect, the difference in the detected tension at the first state and the detected tension at the second state can be used to estimate (e.g., compute, calculate, etc.) a change in volume. In the example, the difference in the detected tension at the first state and the detected tension at the second state can be used to estimate a change in volume of milk as a result of a breastfeeding. In an aspect, the tension detected at the first state can be used to estimate (e.g., compute, calculate, etc.) a first volume. In a further aspect, the tension detected at the second state can be used to estimate (e.g., compute, calculate, etc.) a second volume. In a further aspect, the first estimated volume and the second estimated volume can be used to estimate a change in volume. In an aspect, a caloric intake can be estimated based at least in part on the estimated change in volume and an estimated calories per unit of volume. In the example, the estimated change in volume can be used to estimate the caloric intake as a result of the breastfeeding. In an aspect, the estimated caloric intake can be compared with a target intake to determine an estimated caloric need. In the example, the caloric intake estimated from the breastfeeding can be compared with a target caloric intake. In an aspect, a recommended supplement (e.g., dose, etc.) can be created based at least in part on the comparison of the estimated caloric intake with the target. In the example, a recommendation of some amount of formula can be recommend to supplement the breastfeeding to bring a total caloric intake of a baby to the target caloric intake. Babies should intake a recommended amount of breastmilk and/or calories to avoid malnutrition. The recommended amount of breastmilk can be based on tracking a plurality of babies and/or correlating baby growth to calories and/or consumed breastmilk.

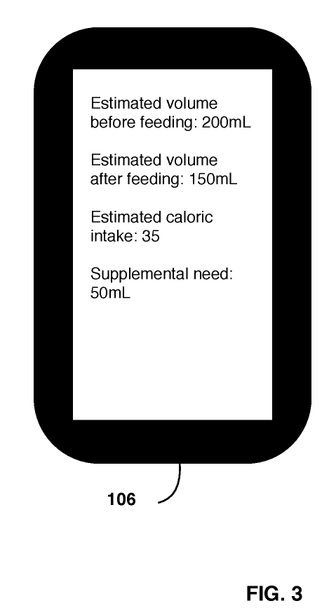

FIG. 3 is an exemplary user interface on a user device 106. The exemplary user interface can display any of the information (e.g., estimations, computations, calculations, etc.) discussed in relation to the descriptions of FIGS. 1 and 2 above. In an aspect, the exemplary user interface can comprise one or more items, such as the detected tension at a first state, the estimated volume at the first state, the detected tension at a second state, the estimated volume at the second state, the change in tension, the estimated change in volume, the estimated caloric intake, the target caloric intake, the estimated caloric need, and/or the recommended supplemental intake. In an aspect, the exemplary user interface can display a normal (for all users) entry and/or a typical (for this user and based at least in part on a historical use for this user) entry for any or all of the one or more items on the exemplary user interface. In an aspect, the normal and/or typical entries can be displayed as a single value and/or a range of values. For example, the exemplary user interface on the user device 106 can comprise the estimated volume before a breastfeeding, the estimated volume after the breastfeeding, an estimated caloric intake, and an estimated volume of formula needed to hit a target caloric intake. In another example, the exemplary user interface on the user device 106 can comprise the estimated change in volume as a result of the breastfeeding, an estimated caloric intake, a target caloric intake, and an estimated volume of formula needed to hit the target caloric intake. In an aspect, an estimated volume can be based on one or more circumferences. In an aspect, an estimated volume can be based on pressure exerted on a fabric. An estimated volume can be compared to a predetermined volume, such as an expected volume.

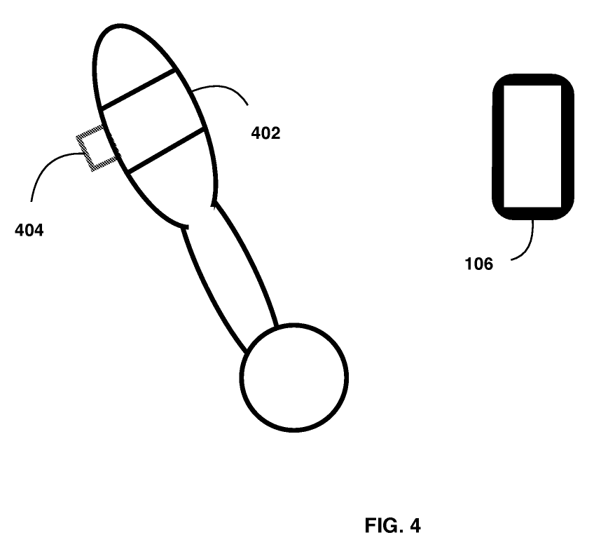

Referring now to FIG. 4, an exemplary system is illustrated. In an aspect, the system can comprise one or more articles of clothing (e.g., fabrics, bras, wraps, sleeves, supports, compression clothing, watches, shirts, pants, etc.) 402. In an aspect, the one or more articles of clothing 402 can comprise a computing device 404. In an aspect, the one or more articles of clothing 402 can comprise material. In an aspect, the computing device 404 can detect tension related to the material of the one or more articles of clothing 402. In an aspect, the computing device 404 can comprise a transmitter. In an aspect, the transmitter can be a transceiver. In an aspect, the computing device 404 can be in communication with a user device 106. In an aspect, the computing device 404 can be in wireless communication with the user device 106. In an aspect, the wireless communication can be Wi-Fi, Bluetooth., etc. In an aspect, the computing device 404 can be in wired communication with the user device 106. In an aspect, the user device 106 can be a smart phone, tablet, laptop, desktop, wearable computing device, etc.

In an aspect, the computing device 404 can represent the detected tension using numbers, units, force, newtons, pounds-force, pounds, etc. In an aspect, the computing device 404 can transmit the detected tension to the user device 106. In an aspect, the computing device 404 can perform one or more evaluations (e.g., computations, etc.) using the detected tension and transmit the results of the one or more computations to the user device 106.

In an aspect, FIG. 4 can illustrate the one or more articles of clothing 402 detecting tension at a first state. As an example, the one or more articles of clothing 402 can be tightly-fitting clothing covering one of more muscle groups 402. In the example, the computing device 404 can detect tension in the clothing 402 when the wearer is in a resting (e.g., not flexed, etc.) state. In an aspect, the computing device 404 can transmit the detected tension to the user device 106, wherein the user device 106 can use the detected tension for one or more evaluations. In another aspect, the computing device 404 can perform one or more evaluations using the detect tension, wherein the computing device 404 can transmit the results of the one or more evaluations to the user device 106. In an aspect, the one or more evaluations can also use historical information. For example, historical information about the tension on the clothing 402 can be used to determine that a density of a target muscle of a user of the clothing 402 is more, less, or substantially consistent with a typical and/or normal density of the muscle. As an example, the current tension on the clothing 402 can indicate a muscle density that is lower or higher than is typical and/or normal. In response to the indication that the muscle density is lower or higher than is typical and/or normal, the user device 106 can receive, create, and/or present a notification. In an aspect, the computing device 404 can create, transmit, and/or present the notification. The notification can be visual, audio, and/or tactile. In an aspect, a user can track one or more diets and/or one or more activities. Diets can comprise food, drinks, nutritional supplements, medication, the like, and/or any combination of the foregoing. Activities can comprise workout routines, workout durations, aerobic activities, anaerobic activities, the like, and/or any combination of the foregoing. A muscle pump can be a difference between a muscle density in a resting position and a muscle density in a working position. In an aspect, a muscle pump based on detected muscle densities can be correlated to the one or more diets and/or the one or more activities for an individual user and/or for an aggregation of multiple users. Additionally, a muscle pump based on detected muscle densities can be correlated to the one or more diets and/or the one or more activities for a muscle, a muscle group, a plurality of muscles, and/or all muscles. A user can indicate a target muscle pump and/or a target change in muscle pump. A diet recommendation and/or an activity recommendation can be made based on the target muscle pump and/or the target change in muscle pump.

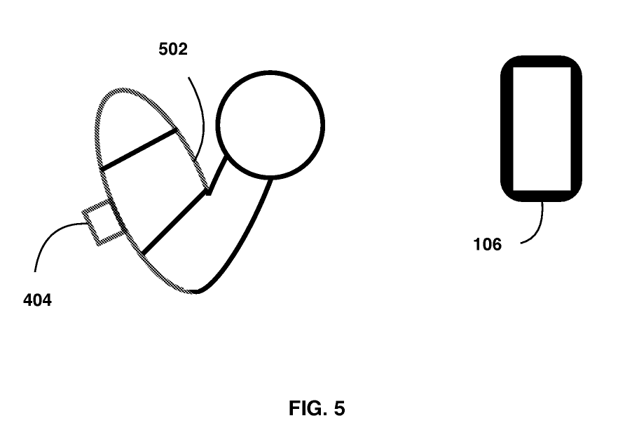

In an aspect, FIG. 5 can illustrate the one or more articles of clothing 502 detecting tension at a second state. As an example, the one or more articles of clothing 502 can be tightly-fitting clothing covering one of more muscle groups 502. In the example, the computing device 404 can detect tension in the clothing 502 when the wearer is in a working (e.g., flexing, etc.) state. In an aspect, the computing device 404 can transmit the detected tension to the user device 106, wherein the user device 106 can use the detected tension for one or more evaluations. In another aspect, the computing device 404 can perform one or more evaluations using the detect tension, wherein the computing device 404 can transmit the results of the one or more evaluations to the user device 106. In an aspect, the one or more evaluations can also use historical information. For example, historical information about the tension on the clothing 502 can be used to determine in the current tension on the clothing 502 indicates that a density of a target muscle of a user of the clothing 502 is more, less, or substantially consistent with a typical and/or normal density of the muscle. As an example, the current tension on the clothing 502 can indicate a muscle density is lower or higher than typical and/or normal. In response to the indication that the muscle density is lower or higher than is typical and/or normal, the user device 106 can receive, create, and/or present a notification. In an aspect, the computing device 404 can create, transmit, and/or present the notification. The notification can be visual, audio, and/or tactile.

In an aspect, the detected tension at the first state can be compared with the detected tension at the second state. In an aspect, the difference in the detected tension at the first state and the detected tension at the second state can be used to estimate (e.g., compute, calculate, etc.) a change in density. In the example, the difference in the detected tension at the first state and the detected tension at the second state can be used to estimate a change in density of muscle during performance of a repetition (rep) of an exercise. In an aspect, the tension detected at the first state can be used to estimate (e.g., compute, calculate, etc.) a first density. In a further aspect, the tension detected at the second state can be used to estimate (e.g., compute, calculate, etc.) a second density. In a further aspect, the first estimated density and the second estimated density can be used to estimate a change in density. In an aspect, a caloric expenditure can be estimated based at least in part on the estimated change in density. The estimated caloric expenditure can be calculated per repetition, per set, per exercise, per muscle group, and/or per workout. In the example, the estimated change in density can be used to estimate the caloric expenditure as a result of exercise. In an aspect, the estimated caloric expenditure can be compared with a target caloric expenditure to determine an estimated caloric expenditure need. In the example, the caloric expenditure estimated based at least in part on the exercise can be compared with a target caloric expenditure. In an aspect, a recommended routine (e.g., exercise, set number repetition number, etc.) can be created based at least in part on the comparison of the estimated caloric expenditure with the target. In the example, a recommendation of some routine can be recommended to bring a total caloric expenditure to the target caloric expenditure. In an aspect, the estimated caloric expenditure can be tracked to determine a caloric intake need. For example, if a user is estimated to expend 400 calories during a routine, then the user can receive a recommendation of 400 calories of protein as part of a post-routine meal. In an aspect, a number of repetitions can be counted based at least in part on the densities. For example, detection of a lower density at 402 can indicate a resting state. Detection of a higher density at 502 can indicate a working state. In an aspect, the number of repetitions can be counted based at least in part on detecting a number of resting states, detecting a number of working states, and/or detecting a number of times a state changes (e.g., from resting to working, from working to resting, etc.). In an aspect, a notification can be created, transmitted, received, and/or presented based at least in part on the counted number of repetitions. For example, when the counted number of repetitions matches a target number of repetitions. In an aspect, an estimated density can be based on one or more circumferences. In an aspect, an estimated density can be based on pressure exerted on a fabric. An estimated density can be compared to a predetermined density, such as an expected density.

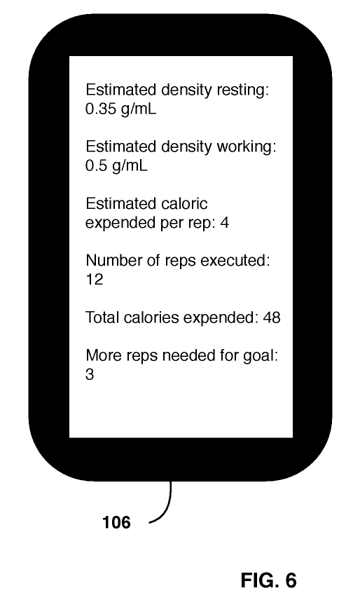

FIG. 6 is an exemplary user interface on a user device 106. The exemplary user interface can display any of the information (e.g., estimations, computations, calculations, etc.) discussed in relation to the descriptions of FIGS. 4 and 5 above. In an aspect, the exemplary user interface can comprise one or more items, such as the detected tension at a first state, the estimated density at the first state, the detected tension at a second state, the estimated density at the second state, the change in tension, the estimated change in density, the estimated caloric expenditure, the target caloric expenditure, the estimated caloric expenditure need, the recommended supplemental intake, the number of repetitions performed, the target number of repetitions, and/or the remaining repetitions needed to be performed to reach the target. In an aspect, the exemplary user interface can display a normal (for all users) entry and/or a typical (for this user and based at least in part on a historical use for this user) entry for any or all of the one or more items on the exemplary user interface. In an aspect, the normal and/or typical entries can be displayed as a single value and/or a range of values. For example, the exemplary user interface on the user device 106 can comprise the estimated resting density, the estimated working density, an estimation of calories per repetition, an estimated number of repetitions, an estimated caloric expenditure, and an estimated number of repetitions remaining to reach a goal.

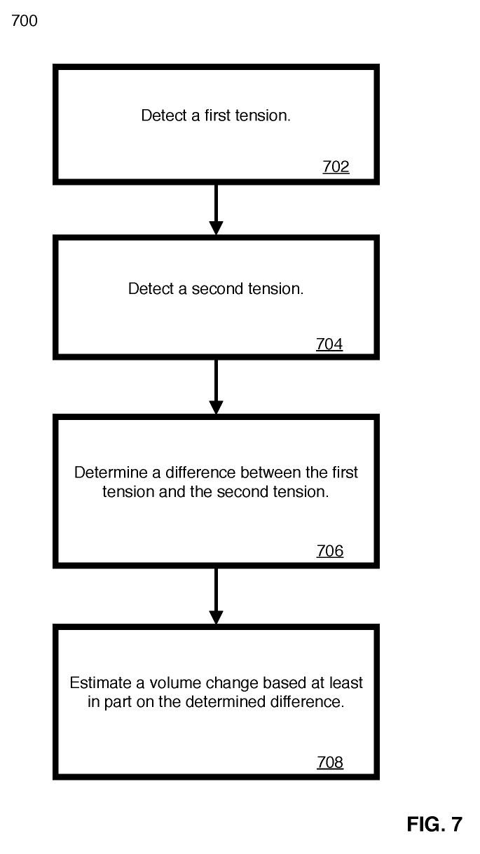

Turning now to FIG. 7, a flow diagram 700 of an exemplary method is illustrated. In an aspect, the exemplary method can be implemented one or more computing devices. At step 702, a first tension can be detected. In an aspect, detecting the first tension can further comprise detecting tension associated with a first portion of fabric. In an aspect, detecting the first tension can further comprise detecting tension associated with a second portion of fabric. In an aspect, detecting the first tension can further comprise detecting tension associated with a third portion of fabric. In an aspect, a bra can comprise the first portion of fabric. In an aspect, the bra can comprise the second portion of fabric. In an aspect, the bra can comprise the third portion of fabric. In an aspect, the first tension can be associated with a pre-breastfeeding state. Although described as comprising three portions of fabric, the bra can comprise one portion of fabric, two portions of fabric, or any number of portions of fabric. Tension in any of the any number of portions of fabric can be detected.

At step 704, a second tension can be detected. In an aspect, detecting the second tension can further comprise detecting tension associated with a first portion of fabric. In an aspect, detecting the second tension can further comprise detecting tension associated with a second portion of fabric. In an aspect, detecting the second tension can further comprise detecting tension associated with a third portion of fabric. In an aspect, a bra can comprise the first portion of fabric. In an aspect, the bra can comprise the second portion of fabric. In an aspect, the bra can comprise the third portion of fabric. In an aspect, the second tension can be associated with a post-breastfeeding state. Although described as comprising three portions of fabric, the bra can comprise one portion of fabric, two portions of fabric, or any number of portions of fabric. Tension in any of the any number of portions of fabric can be detected.

At step 706, a difference between the first tension and the second tension can be determined. At step 708, a volume change can be estimated based at least in part on the determined difference. In an aspect, the estimated volume change can factor in production. Production can be estimated based on known normals for all people and/or certain demographics. Known normals can be stored in a central database and transmitted to the one or more computing devices or a user device in communication with the one or more computing devices. Production can be estimated based on tension changes for a particular user. For example, changes in tension over time can indicate a rate of production. Production can be estimated based on aggregated data on tension changes from a plurality of users. For example, changes in tension over time for a variety of users can be transmitted to a central location. The changes in tension can be transmitted via the one or more computing devices or a user device in communication with the one or more computing devices. Expected production can be computed based on the transmitted changes in tension. The expected production can be transmitted to the one or more computing devices or a user device in communication with the one or more computing devices. In an aspect, the estimated volume change can factor in a production rate. For example, a production rate can change during certain times and/or events, such as during a feeding. Production rates can be estimated for particular times and/or events based on known normals for all people and/or certain demographics. Known normals can be stored in a central database and transmitted to the one or more computing devices or a user device in communication with the one or more computing devices. Production rates can be estimated based on tension changes for a particular user for particular times and/or events. For example, changes in tension changes over time for particular times and/or events can indicate an increase or decrease in rate of production. Production rates for particular times and/or events can be estimated based on aggregated data on tension changes from a plurality of users. For example, changes in tension changes over time for particular times and/or events for a variety of users can be transmitted to a central location. The changes in tension changes can be transmitted via the one or more computing devices or a user device in communication with the one or more computing devices. Expected production rates can be computed based on the transmitted changes in tension changes. The expected production rates can be transmitted to the one or more computing devices or a user device in communication with the one or more computing devices. In an aspect, a caloric intake can be estimated based at least in part on the determined difference. In an aspect, a caloric intake can be estimated based at least in part on the estimated volume change. In an aspect, the estimated caloric intake can be based at least in part on the estimated production. In an aspect, the estimated caloric intake can be based at least in part on the estimated production rate. In an aspect, the estimated caloric intake can be compared to a predetermined value. In an aspect, a recommended supplementation and/or a supplementation amount can be determined based at least in part on the comparison. In an aspect, the recommended supplementation can comprise one or more of: baby formula, breastmilk, liquid food, solid food, any other caloric supplement, the like and/or any combination of the foregoing. In an aspect, any information (e.g., the detected first tension, a first estimated volume, the detected second tension, a second estimated volume, the determined difference between the first tension and the second tension, the estimated volume change, the estimated caloric intake, the comparison of the estimated caloric intake to the predetermined value, the determined recommended supplementation, the determined supplementation amount, the estimated production, the estimated production rate, etc.) can be transmitted to a user device.

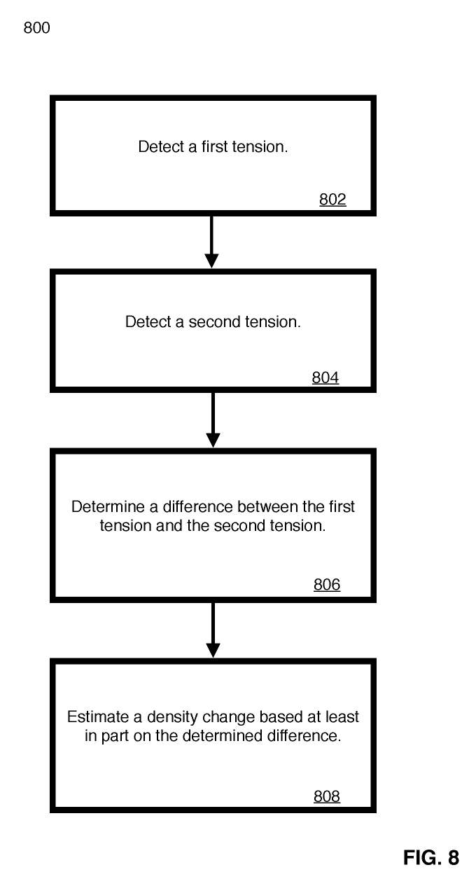

Turning now to FIG. 8, a flow diagram 800 of an exemplary method is illustrated. In an aspect, the exemplary method can be implemented one or more computing devices. At step 802, a first tension can be detected. In an aspect, detecting the first tension can further comprise detecting tension associated with a first portion of fabric. In an aspect, detecting the first tension can further comprise detecting tension associated with a second portion of fabric. In an aspect, detecting the first tension can further comprise detecting tension associated with a third portion of fabric. In an aspect, skin-tight clothing can comprise the first portion of fabric. In an aspect, the skin-tight clothing can comprise the second portion of fabric. In an aspect, the skin-tight clothing can comprise the third portion of fabric. In an aspect, the first portion of fabric can be in communication with one or more muscles. In an aspect, the second portion of fabric can be in communication with one or more muscles. In an aspect, the third portion of fabric can be in communication with one or more muscles. In an aspect, the first tension can be associated with a first position. In an aspect, the first position can be a resting position. Although described as comprising three portions of fabric, the skin-tight clothing can comprise one portion of fabric, two portions of fabric, or any number of portions of fabric. Tension in any of the any number of portions of fabric can be detected.

At step 804, a second tension can be detected. In an aspect, detecting the second tension can further comprise detecting tension associated with a first portion of fabric. In an aspect, detecting the second tension can further comprise detecting tension associated with a second portion of fabric. In an aspect, detecting the second tension can further comprise detecting tension associated with a third portion of fabric. In an aspect, skin-tight clothing can comprise the first portion of fabric. In an aspect, the skin-tight clothing can comprise the second portion of fabric. In an aspect, the skin-tight clothing can comprise the third portion of fabric. In an aspect, the first portion of fabric can be in communication with one or more muscles. In an aspect, the second portion of fabric can be in communication with one or more muscles. In an aspect, the third portion of fabric can be in communication with one or more muscles. In an aspect, the second tension can be associated with a second position. In an aspect, the second position can be a working position. Although described as comprising three portions of fabric, the skin-tight clothing can comprise one portion of fabric, two portions of fabric, or any number of portions of fabric. Tension in any of the any number of portions of fabric can be detected.

At step 806, a difference between the first tension and the second tension can be determined. At step 808, a density change can be estimated based at least in part on the determined difference. In an aspect, the density change can be a change in density associated with one or more muscles. In an aspect, a caloric expenditure can be estimated based at least in part on the determined difference. In an aspect, a caloric expenditure can be estimated based at least in part on the estimated density change. In an aspect, the estimated caloric expenditure can be compared to a predetermined value. In an aspect, a remaining caloric expenditure can be determined based at least in part on the comparison. In an aspect, a recommended supplementation amount can be determined based on the estimated caloric expenditure. In an aspect, the recommended supplementation can comprise one or more of: baby formula, breastmilk, liquid food, solid food, any other caloric supplement, the like and/or any combination of the foregoing. In an aspect, any information (e.g., the detected first tension, a first estimated density, the detected second tension, a second estimated density, the determined difference between the first tension and the second tension, the estimated density change, the estimated caloric expenditure, the comparison of the estimated caloric expenditure to the predetermined value, the determined recommended supplementation, the determined recommended supplementation amount, etc.) can be transmitted to a user device.

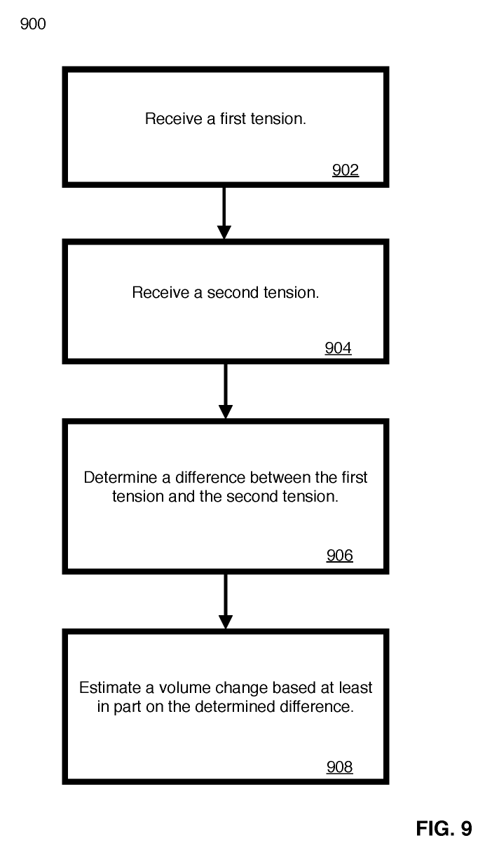

Turning now to FIG. 9, a flow diagram 900 of an exemplary method is illustrated. In an aspect, the exemplary method can be implemented on one or more user devices. At step 902, a first tension can be received. In an aspect, receiving the first tension can further comprise receiving tension associated with a first portion of fabric. In an aspect, receiving the first tension can further comprise receiving tension associated with a second portion of fabric. In an aspect, receiving the first tension can further comprise receiving tension associated with a third portion of fabric. In an aspect, a bra can comprise the first portion of fabric. In an aspect, the bra can comprise the second portion of fabric. In an aspect, the bra can comprise the third portion of fabric. In an aspect, the first tension can be associated with a pre-breastfeeding state. Although described as comprising three portions of fabric, the bra can comprise one portion of fabric, two portions of fabric, or any number of portions of fabric. Tension in any of the any number of portions of fabric can be received.

At step 904, a second tension can be received. In an aspect, receiving the second tension can further comprise receiving tension associated with a first portion of fabric. In an aspect, receiving the second tension can further comprise receiving tension associated with a second portion of fabric. In an aspect, receiving the second tension can further comprise receiving tension associated with a third portion of fabric. In an aspect, a bra can comprise the first portion of fabric. In an aspect, the bra can comprise the second portion of fabric. In an aspect, the bra can comprise the third portion of fabric. In an aspect, the second tension can be associated with a post-breastfeeding state. Although described as comprising three portions of fabric, the bra can comprise one portion of fabric, two portions of fabric, or any number of portions of fabric. Tension in any of the any number of portions of fabric can be received.

At step 906, a difference between the first tension and the second tension can be determined. At step 908, a volume change can be estimated based at least in part on the determined difference. In an aspect, the estimated volume change can factor in production. Production can be estimated based on known normals for all people and/or certain demographics. Known normals can be stored in a central database and transmitted to the one or more user devices. Production can be estimated based on tension changes for a particular user. For example, changes in tension over time can indicate a rate of production. Production can be estimated based on aggregated data on tension changes from a plurality of users. For example, changes in tension over time for a variety of users can be transmitted to a central location. The changes in tension can be transmitted via the one or more user devices. Expected production can be computed based on the transmitted changes in tension. The expected production can be transmitted to the one or more user devices. In an aspect, the estimated volume change can factor in a production rate. For example, a production rate can change during certain times and/or events, such as during a feeding. Production rates can be estimated for particular times and/or events based on known normals for all people and/or certain demographics. Known normals can be stored in a central database and transmitted to the one or more user devices. Production rates can be estimated based on tension changes for a particular user for particular times and/or events. For example, changes in tension changes over time for particular times and/or events can indicate an increase or decrease in rate of production. Production rates for particular times and/or events can be estimated based on aggregated data on tension changes from a plurality of users. For example, changes in tension changes over time for particular times and/or events for a variety of users can be transmitted to a central location. The changes in tension changes can be transmitted via the one or more user devices. Expected production rates can be computed based on the transmitted changes in tension changes. The expected production rates can be transmitted to the one or more user devices. In an aspect, a caloric intake can be estimated based at least in part on the determined difference. In an aspect, a caloric intake can be estimated based at least in part on the estimated volume change. In an aspect, the estimated caloric intake can be based at least in part on the estimated production. In an aspect, the estimated caloric intake can be based at least in part on the estimated production rate. In an aspect, the estimated caloric intake can be compared to a predetermined value. In an aspect, a recommended supplementation and/or a supplementation amount can be determined based at least in part on the comparison. In an aspect, the recommended supplementation can comprise one or more of: baby formula, breastmilk, liquid food, solid food, any other caloric supplement, the like and/or any combination of the foregoing. In an aspect, any information (e.g., the first tension, a first estimated volume, the second tension, a second estimated volume, the determined difference between the first tension and the second tension, the estimated volume change, the estimated caloric intake, the comparison of the estimated caloric intake to the predetermined value, the determined recommended supplementation, the determined supplementation amount, the estimated production, the estimated production rate, etc.) can be received at a user device.

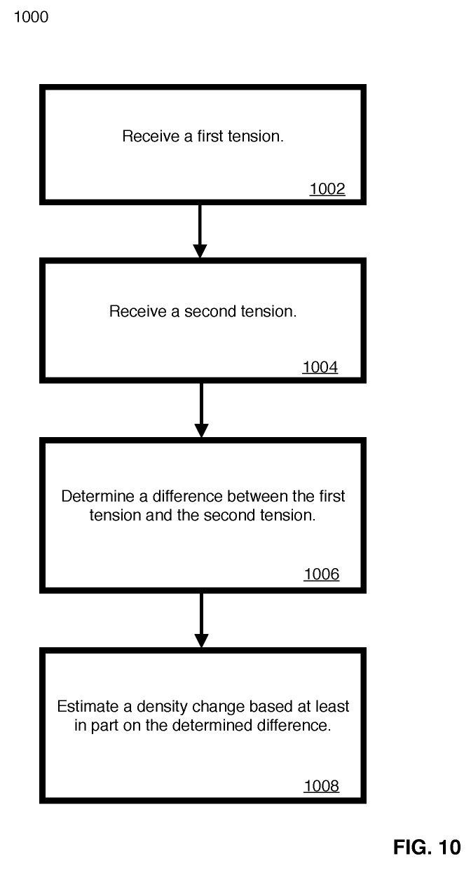

Turning now to FIG. 10, a flow diagram 1000 of an exemplary method is illustrated. In an aspect, the exemplary method can be implemented one or more user devices. At step 1002, a first tension can be received. In an aspect, receiving the first tension can further comprise receiving tension associated with a first portion of fabric. In an aspect, receiving the first tension can further comprise receiving tension associated with a second portion of fabric. In an aspect, receiving the first tension can further comprise receiving tension associated with a third portion of fabric. In an aspect, skin-tight clothing can comprise the first portion of fabric. In an aspect, the skin-tight clothing can comprise the second portion of fabric. In an aspect, the skin-tight clothing can comprise the third portion of fabric. In an aspect, the first portion of fabric can be in communication with one or more muscles. In an aspect, the second portion of fabric can be in communication with one or more muscles. In an aspect, the third portion of fabric can be in communication with one or more muscles. In an aspect, the first tension can be associated with a first position. In an aspect, the first position can be a resting position. Although described as comprising three portions of fabric, the skin-tight clothing can comprise one portion of fabric, two portions of fabric, or any number of portions of fabric. Tension in any of the any number of portions of fabric can be received.

At step 1004, a second tension can be received. In an aspect, receiving the second tension can further comprise receiving tension associated with a first portion of fabric. In an aspect, receiving the second tension can further comprise receiving tension associated with a second portion of fabric. In an aspect, receiving the second tension can further comprise receiving tension associated with a third portion of fabric. In an aspect, skin-tight clothing can comprise the first portion of fabric. In an aspect, the skin-tight clothing can comprise the second portion of fabric. In an aspect, the skin-tight clothing can comprise the third portion of fabric. In an aspect, the first portion of fabric can be in communication with one or more muscles. In an aspect, the second portion of fabric can be in communication with one or more muscles. In an aspect, the third portion of fabric can be in communication with one or more muscles. In an aspect, the second tension can be associated with a second position. In an aspect, the second position can be a working position. Although described as comprising three portions of fabric, the skin-tight clothing can comprise one portion of fabric, two portions of fabric, or any number of portions of fabric. Tension in any of the any number of portions of fabric can be received.

At step 1006, a difference between the first tension and the second tension can be determined. At step 1008, a density change can be estimated based at least in part on the determined difference. In an aspect, the density change can be a change in density associated with one or more muscles. In an aspect, a caloric expenditure can be estimated based at least in part on the determined difference. In an aspect, a caloric expenditure can be estimated based at least in part on the estimated density change. In an aspect, the estimated caloric expenditure can be compared to a predetermined value. In an aspect, a remaining caloric expenditure can be determined based at least in part on the comparison. In an aspect, a recommended supplementation and/or a recommended supplementation amount can be determined based on the estimated caloric expenditure. In an aspect, the recommended supplementation can comprise one or more of: baby formula, breastmilk, liquid food, solid food, any other caloric supplement, the like and/or any combination of the foregoing. In an aspect, any information (e.g., the first tension, a first estimated density, the second tension, a second estimated density, the determined difference between the first tension and the second tension, the estimated density change, the estimated caloric expenditure, the comparison of the estimated caloric expenditure to the predetermined value, the determined recommended supplementation, the determined recommended supplementation amount, etc.) can be received at a user device.

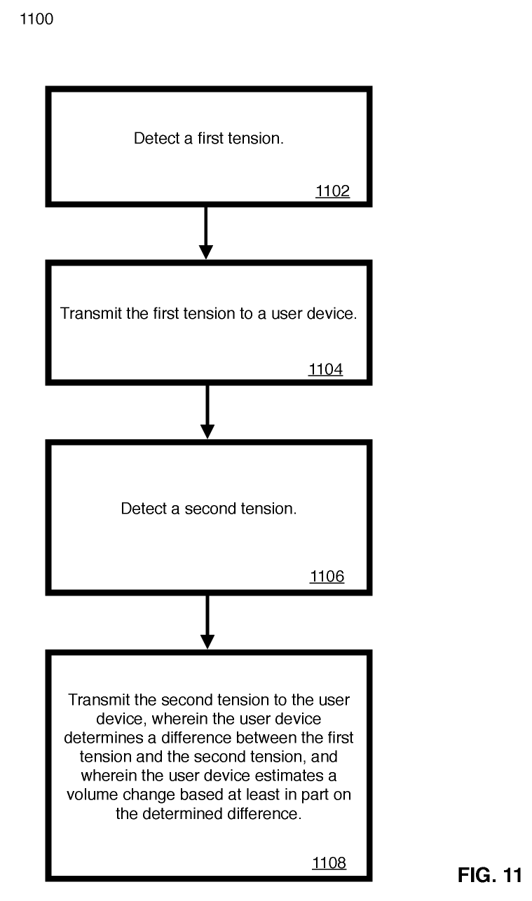

Turning now to FIG. 11, a flow diagram 1100 of an exemplary method is illustrated. In an aspect, the exemplary method can be implemented one or more computing devices. At step 1102, a first tension can be detected. In an aspect, detecting the first tension can further comprise detecting tension associated with a first portion of fabric. In an aspect, detecting the first tension can further comprise detecting tension associated with a second portion of fabric. In an aspect, detecting the first tension can further comprise detecting tension associated with a third portion of fabric. In an aspect, a bra can comprise the first portion of fabric. In an aspect, the bra can comprise the second portion of fabric. In an aspect, the bra can comprise the third portion of fabric. In an aspect, the first tension can be associated with a pre-breastfeeding state. Although described as comprising three portions of fabric, the bra can comprise one portion of fabric, two portions of fabric, or any number of portions of fabric. Tension in any of the any number of portions of fabric can be detected. At step 1104, the first tension can be transmitted to a user device.

At step 1106, a second tension can be detected. In an aspect, detecting the second tension can further comprise detecting tension associated with a first portion of fabric. In an aspect, detecting the second tension can further comprise detecting tension associated with a second portion of fabric. In an aspect, detecting the second tension can further comprise detecting tension associated with a third portion of fabric. In an aspect, a bra can comprise the first portion of fabric. In an aspect, the bra can comprise the second portion of fabric. In an aspect, the bra can comprise the third portion of fabric. In an aspect, the second tension can be associated with a post-breastfeeding state. Although described as comprising three portions of fabric, the bra can comprise one portion of fabric, two portions of fabric, or any number of portions of fabric. Tension in any of the any number of portions of fabric can be detected.

At step 1108, the second tension can be transmitted to the user device. In an aspect, the user device can determine a difference between the first tension and the second tension. In an aspect, the user device can estimate a volume change, based at least in part on the determined difference. In an aspect, the estimated volume change can factor in production. Production can be estimated based on known normals for all people and/or certain demographics. Known normals can be stored in a central database and transmitted to the user device. Production can be estimated based on tension changes for a particular user. For example, changes in tension over time can indicate a rate of production. Production can be estimated based on aggregated data on tension changes from a plurality of users. For example, changes in tension over time for a variety of users can be transmitted to a central location. The changes in tension can be transmitted via the user device. Expected production can be computed based on the transmitted changes in tension. The expected production can be transmitted to the user device. In an aspect, the estimated volume change can factor in a production rate. For example, a production rate can change during certain times and/or events, such as during a feeding. Production rates can be estimated for particular times and/or events based on known normals for all people and/or certain demographics. Known normals can be stored in a central database and transmitted to the user device. Production rates can be estimated based on tension changes for a particular user for particular times and/or events. For example, changes in tension changes over time for particular times and/or events can indicate an increase or decrease in rate of production. Production rates for particular times and/or events can be estimated based on aggregated data on tension changes from a plurality of users. For example, changes in tension changes over time for particular times and/or events for a variety of users can be transmitted to a central location. The changes in tension changes can be transmitted via the user device. Expected production rates can be computed based on the transmitted changes in tension changes. The expected production rates can be transmitted to the user device. In an aspect, a caloric intake can be estimated based at least in part on the determined difference. In an aspect, a caloric intake can be estimated based at least in part on the estimated volume change. In an aspect, the estimated caloric intake can be based at least in part on the estimated production. In an aspect, the estimated caloric intake can be based at least in part on the estimated production rate. In an aspect, the estimated caloric intake can be compared to a predetermined value. In an aspect, a recommended supplementation and/or a supplementation amount can be determined based at least in part on the comparison. In an aspect, the recommended supplementation can comprise one or more of: baby formula, breastmilk, liquid food, solid food, any other caloric supplement, the like and/or any combination of the foregoing.

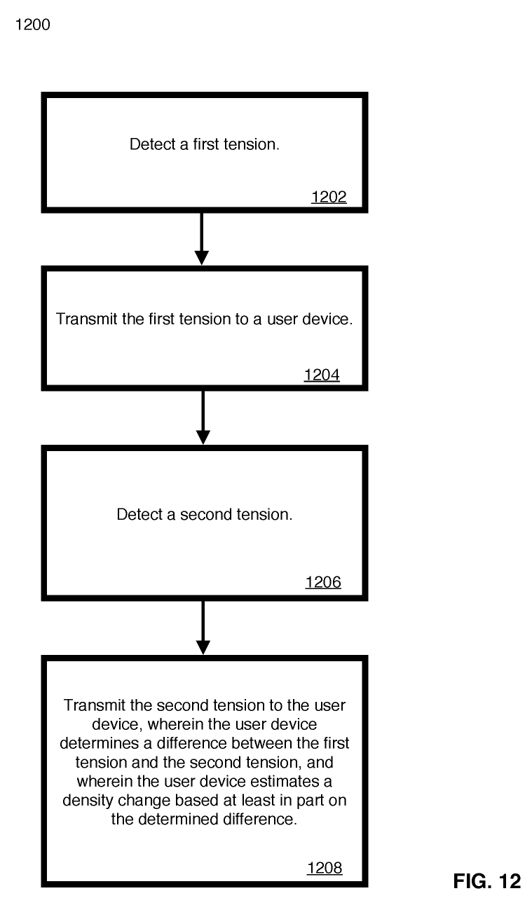

Turning now to FIG. 12, a flow diagram 1200 of an exemplary method is illustrated. In an aspect, the exemplary method can be implemented one or more computing devices. At step 1202, a first tension can be detected. In an aspect, detecting the first tension can further comprise detecting tension associated with a first portion of fabric. In an aspect, detecting the first tension can further comprise detecting tension associated with a second portion of fabric. In an aspect, detecting the first tension can further comprise detecting tension associated with a third portion of fabric. In an aspect, skin-tight clothing can comprise the first portion of fabric. In an aspect, the skin-tight clothing can comprise the second portion of fabric. In an aspect, the skin-tight clothing can comprise the third portion of fabric. In an aspect, the first portion of fabric can be in communication with one or more muscles. In an aspect, the second portion of fabric can be in communication with one or more muscles. In an aspect, the third portion of fabric can be in communication with one or more muscles. In an aspect, the first tension can be associated with a first position. In an aspect, the first position can be a resting position. Although described as comprising three portions of fabric, the skin-tight clothing can comprise one portion of fabric, two portions of fabric, or any number of portions of fabric. Tension in any of the any number of portions of fabric can be detected. At step 1204, the first tension can be transmitted to a user device.

At step 1206, a second tension can be detected. In an aspect, detecting the second tension can further comprise detecting tension associated with a first portion of fabric. In an aspect, detecting the second tension can further comprise detecting tension associated with a second portion of fabric. In an aspect, detecting the second tension can further comprise detecting tension associated with a third portion of fabric. In an aspect, skin-tight clothing can comprise the first portion of fabric. In an aspect, the skin-tight clothing can comprise the second portion of fabric. In an aspect, the skin-tight clothing can comprise the third portion of fabric. In an aspect, the first portion of fabric can be in communication with one or more muscles. In an aspect, the second portion of fabric can be in communication with one or more muscles. In an aspect, the third portion of fabric can be in communication with one or more muscles. In an aspect, the second tension can be associated with a second position. In an aspect, the second position can be a working position. Although described as comprising three portions of fabric, the skin-tight clothing can comprise one portion of fabric, two portions of fabric, or any number of portions of fabric. Tension in any of the any number of portions of fabric can be detected.

At step 1208, the second tension can be transmitted to the user device. In an aspect, the user device can determine a difference between the first tension and the second tension. In an aspect, the user device can estimate a density change, based at least in part on the determined difference. In an aspect, the density change can be a change in density associated with one or more muscles. In an aspect, a caloric expenditure can be estimated based at least in part on the determined difference. In an aspect, a caloric expenditure can be estimated based at least in part on the estimated density change. In an aspect, the estimated caloric expenditure can be compared to a predetermined value. In an aspect, a remaining caloric expenditure can be determined based at least in part on the comparison. In an aspect, a recommended supplementation and/or a recommended supplementation amount can be determined based on the estimated caloric expenditure. In an aspect, the recommended supplementation can comprise one or more of: baby formula, breastmilk, liquid food, solid food, any other caloric supplement, the like and/or any combination of the foregoing.

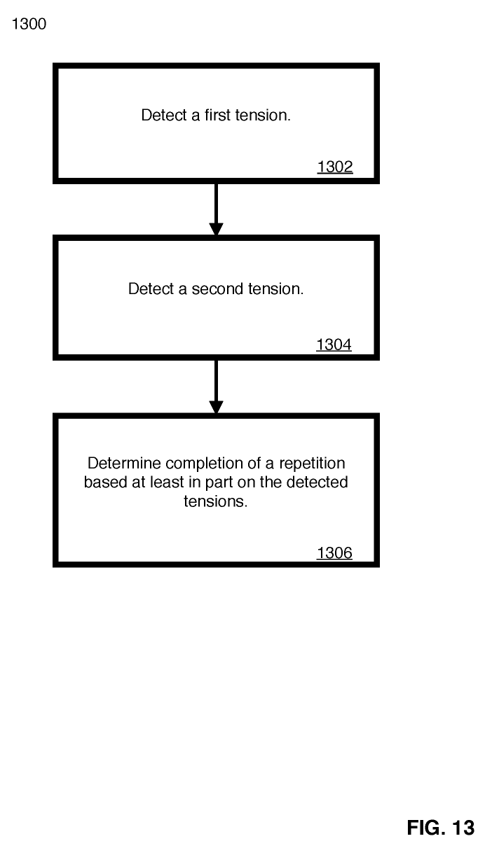

Turning now to FIG. 13, a flow diagram 1300 of an exemplary method is illustrated. In an aspect, the exemplary method can be implemented one or more computing devices. At step 1302, a first tension can be detected. In an aspect, detecting the first tension can further comprise detecting tension associated with a first portion of fabric. In an aspect, detecting the first tension can further comprise detecting tension associated with a second portion of fabric. In an aspect, detecting the first tension can further comprise detecting tension associated with a third portion of fabric. In an aspect, skin-tight clothing can comprise the first portion of fabric. In an aspect, the skin-tight clothing can comprise the second portion of fabric. In an aspect, the skin-tight clothing can comprise the third portion of fabric. In an aspect, the first portion of fabric can be in communication with one or more muscles. In an aspect, the second portion of fabric can be in communication with one or more muscles. In an aspect, the third portion of fabric can be in communication with one or more muscles. In an aspect, the first tension can be associated with a first position. In an aspect, the first position can be a resting position. Although described as comprising three portions of fabric, the skin-tight clothing can comprise one portion of fabric, two portions of fabric, or any number of portions of fabric. Tension in any of the any number of portions of fabric can be detected.

At step 1304, a second tension can be detected. In an aspect, detecting the second tension can further comprise detecting tension associated with a first portion of fabric. In an aspect, detecting the second tension can further comprise detecting tension associated with a second portion of fabric. In an aspect, detecting the second tension can further comprise detecting tension associated with a third portion of fabric. In an aspect, skin-tight clothing can comprise the first portion of fabric. In an aspect, the skin-tight clothing can comprise the second portion of fabric. In an aspect, the skin-tight clothing can comprise the third portion of fabric. In an aspect, the first portion of fabric can be in communication with one or more muscles. In an aspect, the second portion of fabric can be in communication with one or more muscles. In an aspect, the third portion of fabric can be in communication with one or more muscles. In an aspect, the second tension can be associated with a second position. In an aspect, the second position can be a working position. Although described as comprising three portions of fabric, the skin-tight clothing can comprise one portion of fabric, two portions of fabric, or any number of portions of fabric. Tension in any of the any number of portions of fabric can be detected.

At step 1306, completion of a repetition can be determined based at least in part on the determined tensions. In an aspect, the repetition can be one of a plurality of repetitions associated with a set. In an aspect, the set can be one of a plurality of sets associated with an exercise. In an aspect, the exercise can be one of a plurality of exercises associated with a muscle and/or muscle group. In an aspect, muscle and or muscle group can be one of a plurality of muscles and/or muscle groups associated with a workout. In an aspect, a repetition count can be increased (e.g., incremented, etc.) based on the determined completion of the repetition. In an aspect, the repetition count can be compared to a predetermined value (e.g., a target repetition, etc.). In an aspect, if the repetition is a final repetition of a set, then a set count can be increased (e.g., incremented, etc.) and the repetition count can reset (e.g., be set to zero, be set to one, be set to a number associated with a total number of repetitions in a set to be performed, etc.). In an aspect, if the repetition is a final repetition of a final set of an exercise, then a new exercise can be selected (e.g., chosen, etc.), the set count can be reset (e.g., be set to zero, be set to one, be set to a number associated with a total number of sets of the new exercise to be performed, etc.), and the repetition count can be reset (e.g., be set to zero, be set to one, be set to a number associated with a total number of repetitions in a set to be performed, etc.). In an aspect, if the repetition is a final repetition of a final set of a final exercise for a muscle or muscle group, then a new muscle or muscle group can be selected (e.g., chosen, etc.), a new exercise associated with new muscle or muscle group can be selected (e.g., chosen, etc.), the set count can be reset (e.g., be set to zero, be set to one, be set to a number associated with a total number of sets of the new exercise to be performed, etc.), and the repetition count can be reset (e.g., be set to zero, be set to one, be set to a number associated with a total number of repetitions in a set to be performed, etc.). In an aspect, a remaining repetitions can be determined based on the comparison of the repetition count to the predetermined value. In an aspect, a caloric expenditure can be estimated based one or more of the determined repetition, a total repetitions associated with a set, a total repetitions associated with an exercise, a total repetitions associated with a muscle or muscle group, a total repetitions associated with a workout, the repetition count, the set count, or a combination thereof. In an aspect, the estimated caloric expenditure can be compared to a predetermined value (e.g., a target caloric expenditure, etc.). In an aspect, a remaining caloric expenditure can be determined based at least in part on the comparison. In an aspect, a recommended supplementation and/or a recommended supplementation amount can be determined based on the estimated caloric expenditure. In an aspect, the recommended supplementation can comprise one or more of: baby formula, breastmilk, liquid food, solid food, any other caloric supplement, the like and/or any combination of the foregoing. In an aspect, any information (e.g., the detected first tension, a first estimated density, the detected second tension, a second estimated density, the repetition count, the set count, the new exercise, the new muscle or muscle group, the estimated caloric expenditure, the comparison of the estimated caloric expenditure to the predetermined value, the determined recommended supplementation, the determined recommended supplementation amount, etc.) can be transmitted to a user device.

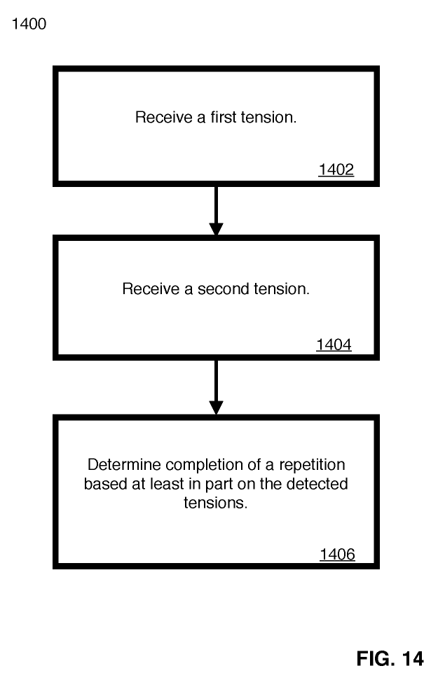

Turning now to FIG. 14, a flow diagram 1400 of an exemplary method is illustrated. In an aspect, the exemplary method can be implemented one or more user devices. At step 1402, a first tension can be received. In an aspect, receiving the first tension can further comprise receiving tension associated with a first portion of fabric. In an aspect, receiving the first tension can further comprise receiving tension associated with a second portion of fabric. In an aspect, receiving the first tension can further comprise receiving tension associated with a third portion of fabric. In an aspect, skin-tight clothing can comprise the first portion of fabric. In an aspect, the skin-tight clothing can comprise the second portion of fabric. In an aspect, the skin-tight clothing can comprise the third portion of fabric. In an aspect, the first portion of fabric can be in communication with one or more muscles. In an aspect, the second portion of fabric can be in communication with one or more muscles. In an aspect, the third portion of fabric can be in communication with one or more muscles. In an aspect, the first tension can be associated with a first position. In an aspect, the first position can be a resting position. Although described as comprising three portions of fabric, the skin-tight clothing can comprise one portion of fabric, two portions of fabric, or any number of portions of fabric. Tension in any of the any number of portions of fabric can be received.

At step 1404, a second tension can be received. In an aspect, receiving the second tension can further comprise receiving tension associated with a first portion of fabric. In an aspect, receiving the second tension can further comprise receiving tension associated with a second portion of fabric. In an aspect, receiving the second tension can further comprise receiving tension associated with a third portion of fabric. In an aspect, skin-tight clothing can comprise the first portion of fabric. In an aspect, the skin-tight clothing can comprise the second portion of fabric. In an aspect, the skin-tight clothing can comprise the third portion of fabric. In an aspect, the first portion of fabric can be in communication with one or more muscles. In an aspect, the second portion of fabric can be in communication with one or more muscles. In an aspect, the third portion of fabric can be in communication with one or more muscles. In an aspect, the second tension can be associated with a second position. In an aspect, the second position can be a working position. Although described as comprising three portions of fabric, the skin-tight clothing can comprise one portion of fabric, two portions of fabric, or any number of portions of fabric. Tension in any of the any number of portions of fabric can be received.

At step 1406, completion of a repetition can be determined based at least in part on the determined tensions. In an aspect, the repetition can be one of a plurality of repetitions associated with a set. In an aspect, the set can be one of a plurality of sets associated with an exercise. In an aspect, the exercise can be one of a plurality of exercises associated with a muscle and/or muscle group. In an aspect, muscle and or muscle group can be one of a plurality of muscles and/or muscle groups associated with a workout. In an aspect, a repetition count can be increased (e.g., incremented, etc.) based on the determined completion of the repetition. In an aspect, the repetition count can be compared to a predetermined value (e.g., a target repetition, etc.). In an aspect, if the repetition is a final repetition of a set, then a set count can be increased (e.g., incremented, etc.) and the repetition count can reset (e.g., be set to zero, be set to one, be set to a number associated with a total number of repetitions in a set to be performed, etc.). In an aspect, if the repetition is a final repetition of a final set of an exercise, then a new exercise can be selected (e.g., chosen, etc.), the set count can be reset (e.g., be set to zero, be set to one, be set to a number associated with a total number of sets of the new exercise to be performed, etc.), and the repetition count can be reset (e.g., be set to zero, be set to one, be set to a number associated with a total number of repetitions in a set to be performed, etc.). In an aspect, if the repetition is a final repetition of a final set of a final exercise for a muscle or muscle group, then a new muscle or muscle group can be selected (e.g., chosen, etc.), a new exercise associated with new muscle or muscle group can be selected (e.g., chosen, etc.), the set count can be reset (e.g., be set to zero, be set to one, be set to a number associated with a total number of sets of the new exercise to be performed, etc.), and the repetition count can be reset (e.g., be set to zero, be set to one, be set to a number associated with a total number of repetitions in a set to be performed, etc.). In an aspect, a remaining repetitions can be determined based on the comparison of the repetition count to the predetermined value. In an aspect, a caloric expenditure can be estimated based one or more of the determined repetition, a total repetitions associated with a set, a total repetitions associated with an exercise, a total repetitions associated with a muscle or muscle group, a total repetitions associated with a workout, the repetition count, the set count, or a combination thereof. In an aspect, the estimated caloric expenditure can be compared to a predetermined value (e.g., a target caloric expenditure, etc.). In an aspect, a remaining caloric expenditure can be determined based at least in part on the comparison. In an aspect, a recommended supplementation and/or a recommended supplementation amount can be determined based on the estimated caloric expenditure. In an aspect, the recommended supplementation can comprise one or more of: baby formula, breastmilk, liquid food, solid food, any other caloric supplement, the like and/or any combination of the foregoing. In an aspect, any information (e.g., the first tension, a first estimated density, the second tension, a second estimated density, the repetition count, the set count, the new exercise, the new muscle or muscle group, the estimated caloric expenditure, the comparison of the estimated caloric expenditure to the predetermined value, the determined recommended supplementation, the determined recommended supplementation amount, etc.) can be received at a user device.

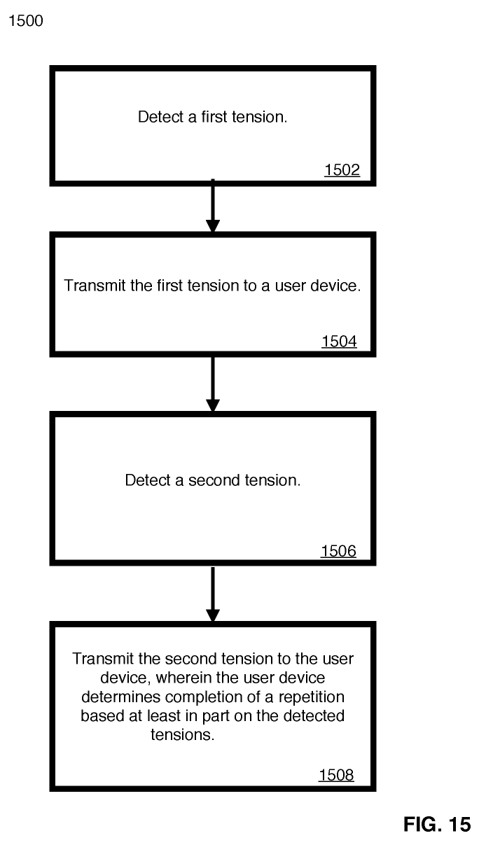

Turning now to FIG. 15, a flow diagram 1500 of an exemplary method is illustrated. In an aspect, the exemplary method can be implemented one or more computing devices. At step 1502, a first tension can be detected. In an aspect, detecting the first tension can further comprise detecting tension associated with a first portion of fabric. In an aspect, detecting the first tension can further comprise detecting tension associated with a second portion of fabric. In an aspect, detecting the first tension can further comprise detecting tension associated with a third portion of fabric. In an aspect, skin-tight clothing can comprise the first portion of fabric. In an aspect, the skin-tight clothing can comprise the second portion of fabric. In an aspect, the skin-tight clothing can comprise the third portion of fabric. In an aspect, the first portion of fabric can be in communication with one or more muscles. In an aspect, the second portion of fabric can be in communication with one or more muscles. In an aspect, the third portion of fabric can be in communication with one or more muscles. In an aspect, the first tension can be associated with a first position. In an aspect, the first position can be a resting position. Although described as comprising three portions of fabric, the skin-tight clothing can comprise one portion of fabric, two portions of fabric, or any number of portions of fabric. Tension in any of the any number of portions of fabric can be detected. At step 1504, the first tension can be transmitted to a user device.

At step 1506, a second tension can be detected. In an aspect, detecting the second tension can further comprise detecting tension associated with a first portion of fabric. In an aspect, detecting the second tension can further comprise detecting tension associated with a second portion of fabric. In an aspect, detecting the second tension can further comprise detecting tension associated with a third portion of fabric. In an aspect, skin-tight clothing can comprise the first portion of fabric. In an aspect, the skin-tight clothing can comprise the second portion of fabric. In an aspect, the skin-tight clothing can comprise the third portion of fabric. In an aspect, the first portion of fabric can be in communication with one or more muscles. In an aspect, the second portion of fabric can be in communication with one or more muscles. In an aspect, the third portion of fabric can be in communication with one or more muscles. In an aspect, the second tension can be associated with a second position. In an aspect, the second position can be a working position. Although described as comprising three portions of fabric, the skin-tight clothing can comprise one portion of fabric, two portions of fabric, or any number of portions of fabric. Tension in any of the any number of portions of fabric can be detected.

At step 1508, the second tension can be transmitted to the user device. The user device can determine completion of a repetition based at least in part on the determined tensions. In an aspect, the repetition can be one of a plurality of repetitions associated with a set. In an aspect, the set can be one of a plurality of sets associated with an exercise. In an aspect, the exercise can be one of a plurality of exercises associated with a muscle and/or muscle group. In an aspect, muscle and or muscle group can be one of a plurality of muscles and/or muscle groups associated with a workout. In an aspect, a repetition count can be increased (e.g., incremented, etc.) based on the determined completion of the repetition. In an aspect, the repetition count can be compared to a predetermined value (e.g., a target repetition, etc.). In an aspect, if the repetition is a final repetition of a set, then a set count can be increased (e.g., incremented, etc.) and the repetition count can reset (e.g., be set to zero, be set to one, be set to a number associated with a total number of repetitions in a set to be performed, etc.). In an aspect, if the repetition is a final repetition of a final set of an exercise, then a new exercise can be selected (e.g., chosen, etc.), the set count can be reset (e.g., be set to zero, be set to one, be set to a number associated with a total number of sets of the new exercise to be performed, etc.), and the repetition count can be reset (e.g., be set to zero, be set to one, be set to a number associated with a total number of repetitions in a set to be performed, etc.). In an aspect, if the repetition is a final repetition of a final set of a final exercise for a muscle or muscle group, then a new muscle or muscle group can be selected (e.g., chosen, etc.), a new exercise associated with new muscle or muscle group can be selected (e.g., chosen, etc.), the set count can be reset (e.g., be set to zero, be set to one, be set to a number associated with a total number of sets of the new exercise to be performed, etc.), and the repetition count can be reset (e.g., be set to zero, be set to one, be set to a number associated with a total number of repetitions in a set to be performed, etc.). In an aspect, a remaining repetitions can be determined based on the comparison of the repetition count to the predetermined value. In an aspect, a caloric expenditure can be estimated based one or more of the determined repetition, a total repetitions associated with a set, a total repetitions associated with an exercise, a total repetitions associated with a muscle or muscle group, a total repetitions associated with a workout, the repetition count, the set count, or a combination thereof. In an aspect, the estimated caloric expenditure can be compared to a predetermined value (e.g., a target caloric expenditure, etc.). In an aspect, a remaining caloric expenditure can be determined based at least in part on the comparison. In an aspect, a recommended supplementation and/or a recommended supplementation amount can be determined based on the estimated caloric expenditure. In an aspect, the recommended supplementation can comprise one or more of: baby formula, breastmilk, liquid food, solid food, any other caloric supplement, the like and/or any combination of the foregoing.

Accordingly, the present invention may be realized in hardware, software, or a combination of hardware and software. The present invention may be realized in a centralized fashion in at least one computer system, or in a distributed fashion where different elements are spread across several interconnected computer systems. Any kind of computer system or other apparatus, including smart phones and other mobile devices, adapted for carrying out the methods described herein is suited. A typical combination of hardware and software with a computer system with a computer program that, when being loaded and executed, controls the computer system such that it carries out the methods described herein. Another typical combination may be a general-purpose computer system with a computer program that, when being loaded and executed, controls the computer system such that it carries out the methods described herein. Another typical combination may be both a mobile communication device with a computer system with a computer program that, when being loaded and executed, controls the computer system such that it carriers out the methods described herein and a general-purpose computer system with a computer program that, when being loaded and executed, controls the computer system such that it carries out the methods described herein.

The present invention may also be embedded in a computer program product, which comprises all the features enabling the implementation of the methods described herein, and which when loaded in a computer system is able to carry out these methods. Computer program in the present context means any expression, in any language, code or notation, of a set of instructions intended to cause a system having an information processing capability to perform a particular function either directly or after either or both of the following: a) conversion to another language, code or notation; b) reproduction in a different material form.

While the present invention has been described with reference to certain embodiments, it will be understood by those skilled in the art that various changes may be made and equivalents may be substituted without departing from the scope of the present invention. In addition, many modifications may be made to adapt a particular situation or material to the teachings of the present invention without departing from its scope. Therefore, it is intended that the present invention not be limited to the particular embodiment disclosed, but that the present invention will include all embodiments falling within the scope of the appended claims.

What is claimed is:

1. A method implemented on one or more computing device comprising:
detecting a first tension;
detecting a second tension;
determining a difference between the first tension and the second tension; and
estimating a volume change based at least in part on the determined difference.

2. The method of claim 1, further comprising:
estimating a caloric intake based at least in part on the estimated volume change.

3. The method of claim 2, further comprising:
transmitting the estimated caloric intake to a user device.

4. The method of claim 2, further comprising:
comparing the estimated caloric intake to a predetermined value.

5. The method of claim 4, further comprising:
determining a recommended supplementation based at least in part on the comparison.

6. The method of claim 5, wherein the recommended supplementation comprises one or more of: baby formula, breastmilk, liquid food, and solid food.

7. The method of claim 1, wherein the detecting the first tension further comprises detecting tension associated with a first portion of fabric.

8. The method of claim 7, wherein a bra comprises the first portion of the fabric.

9. The method of claim 1, wherein the first tension is associated with a pre-breastfeeding state.

10. The method of claim 1, wherein the second tension is associated with a post-breastfeeding state.

11. A method implemented on one or more computing device comprising:
detecting a first tension;
detecting a second tension;
determining a difference between the first tension and the second tension; and
estimating a density change based at least in part on the determined difference.

12. The method of claim 11, wherein the first tension is associated with a first position.

13. The method of claim 12, wherein the first position is a resting position.

14. The method of claim 12, wherein the second tension is associated with a second position.

15. The method of claim 14, wherein the second position is a working position.

16. The method of claim 11, further comprising:
estimating a caloric expenditure based at least in part on the estimated density change.

17. The method of claim 11, wherein the density change is a change in density associated with one or more muscles.

18. The method of claim 17, wherein the detecting the first tension further comprises detecting tension associated with a first portion of fabric.

19. The method of claim 11, further comprising:
transmitting the density change to a user device.

20. A method implemented on one or more computing device comprising:
detecting a first tension;
detecting a second tension; and
determining completion of a repetition based at least in part on the detected tensions.
